(19) United States
(12) Patent Application Publication
(10) Pub. No.: US 2010/0249027 A1
(43) Pub. Date: Sep. 30, 2010

(54) CRF CONJUGATES WITH EXTENDED HALF-LIVES

(76) Inventor: William Henry, Haddenham (GB)

Correspondence Address: JONES DAY, 222 EAST 41ST ST, NEW YORK, NY, 10017, US

(21) Appl. No.: 12/601,714

(22) PCT Filed: May 27, 2008
(86) PCT No.: PCT/IB08/03167

§ 371 Date: May 27, 2010

Related U.S. Application Data
(60) Provisional application No. 60/931,786, filed on May 25, 2007.

(51) Int. Cl. A61K 38/35; C07K 14/00; A61P 7/10

(52) U.S. Cl. 514/10.8; 530/306

(57) ABSTRACT

The present invention relates to conjugates of CRF that have been modified to include a moiety that protects CRF from degradation and prolongs the half-life of CRF. The CRF conjugates of the invention have an increased half-life which results in a dose-sparing effect and less frequent administration

### 1. FIELD OF INVENTION

The invention relates to conjugates of corticotropin-releasing factor (CRF) having an increased half-life and stability as compared to unmodified CRF.

### 2. BACKGROUND OF THE INVENTION

Corticotropin-Releasing Factor (CRF) is an endogenous 41 amino acid peptide first identified in 1981 as the major hypothalamic hormone responsible for stimulation of the pituitary-adrenal axis (Vale, W., et al., Science 213:1394-1397 (1981)). CRF can be obtained from natural sources, expressed recombinantly, or produced synthetically.

CRF has been shown to have a peripheral, non-endocrine function mediated biological activity as a potent inhibitor of edema and inflammation (Wei, E. T. et al., Ciba Foundation Symposium 172:258-276 (1993)). This has been confirmed in a series of experiments in which systemic administration of CRF has been shown to inhibit vascular leakage of plasma constituents and associated tissue swelling in response to injury or inflammatory mediators (Wei, E. T. et al., European J. of Pharm. 140:63-67 (1987), Serda, S. M. et al., Pharm. Res. 26:85-91 (1992) and Wei, E. T. et al., Regulatory Peptides 33:93-104 (1991)). CRF is also known in the art as corticotrop(h)in-releasing hormone (CRH), corticoliberin, corticorelin and CRF-41.

The CRF neuropeptide was first isolated from extracts of ovine hypothalami (OCRF; Vale, W., et al., Science 213:1394-1397 (1981)) and has subsequently been identified and isolated from the hypothalamus of numerous other mammals including rat (rCRF; Rivier, J., et al., Proc. Natl. Acad. Sci. USA 80:4851-4855 (1983)), porcine (PCRF; Schally, A., et al., Proc. Natl. Acad. Sci. USA 78:5197-5201 (1981) and human (hCRF; Shibahara, S., et al., EMBO J. 2:775-779 (1983)). Comparison of the amino acid sequences of CRF peptides from ovine, rat and human has shown that the rat and human peptides are identical, both differing at seven amino acid positions from the ovine peptide, the differences occurring largely in the C-terminal region of the peptides (Hermus, A., et al., J. Clin. Endocrin. and Metabolism 58:187-191 (1984) and Saphier, P., et al., J. Endocrin. 133:487-495 (1993)).

CRF has been shown to be a safe and useful pharmaceutical agent for a variety of different applications in humans. Specifically, in vivo administration of CRF has been extensively employed to help elucidate the cause of hyper- and hypo-cortisolemic conditions in humans and is an extremely useful diagnostic and investigative tool for various other disorders affecting the hypothalamic-pituitary-adrenal axis, including endogenous depression and Cushing's disease (Chrousos, G., et al., N. Eng. J. Med. 310:622 (1984) and Lytras, N., et al., Clin. Endocrinol. 20:71 (1984)). In fact, in vivo administration of CRF is useful to test corticotropic function of the anterior pituitary in all cases in which an impairment of the anterior pituitary function is suspected. This applies to patients with pituitary tumors or craniopharyngiomas, patients with suspected pituitary insufficiency, panhypopituitarism or empty sella syndrome, as well as patients with traumatic or post-operative injury to the pituitary region and patients who have undergone radiotherapy of the pituitary region. Thus, CRF clearly has utility for diagnostic analysis of the hypothalamus-pituitary-adrenal (HPA) axis.

For important peripheral applications, CRF also possesses in vivo anti-inflammatory activity. With regard to the anti-inflammatory activity of the CRF peptide, CRF prevents vascular leakage induced by a variety of inflammatory mediators that appear to act selectively on post-capillary venules in skin. CRF also inhibits injury- and inflammatory mediator-induced leakage from capillaries in muscle, cerebral micro-vessels, and lung alveolar capillaries. These observations suggest that CRF acts throughout the micro-circulation to preserve or restore endothelial cell integrity, thereby inhibiting fluid egress and white blood cell trafficking from the intravascular space and accumulation at sites of injury.

In light of the novel anti-inflammatory activity of the CRF peptide, numerous clinical indications are evident. For example, clinical indications for which the CRF peptide may find use include rheumatoid arthritis, edema secondary to brain tumors or irradiation for cancer, edema resulting from stroke, head trauma or spinal cord injury, post-surgical edema, asthma and other respiratory diseases and cystoid macular edema of the eye.

One of the challenges of many polypeptides used in disease treatment is that they have a relatively short half-life after administration. Proteins introduced into the blood are rapidly cleared from the mammalian subject by the kidneys. This is especially a problem in lower molecular weight polypeptides, such as CRF. Therefore, many polypeptide therapies require higher dosages or require shorter time periods between dosing to have their desired effect. Common approaches to extending the circulation half-life of therapeutic compounds is to encase them in liposomes, link proteins to human or bovine serum albumin, or synthesize polymer conjugates of the active protein. Citation of any reference in Section 2 of this application is not an admission that the reference is prior art to the application.

### 3. SUMMARY OF THE INVENTION

The present invention relates to conjugates of CRF that have been modified to include a moiety that protects CRF from degradation and prolongs the half-life of CRF. The CRF conjugates of the invention have an increased half-life which results in a dose-sparing effect and less frequent administration. An example of a CRF conjugate is CRF that has been modified to include moieties such as polyethylene glycol covalently bound to CRF.

In one embodiment, the invention provides for CRF conjugates comprising CRF wherein said CRF is chemically modified with polyethylene glycol. In another embodiment, the CRF component of the CRF conjugate has the sequence identified as human CRF identified in FIG. 1. Alternatively, the sequence of CRF may be modified or derivatized to include one or more changes in the amino acid sequence, including, but not limited to insertions, deletions or substitutions. In yet another embodiment the sequence of CRF has been modified to include one or more cysteine residues. The sequence of CRF may include cysteine as a substitution of one or more of the existing residues of CRF, alternatively, the cysteine residue may be incorporated as an addition to the existing sequence of CRF. The cysteine residues may be inserted within the sequence of CRF, or the cysteine residue may be added to the amino or carboxy terminus of the sequence. In another embodiment, cysteine residues are added to the amino and carboxy termini of the sequence. When two or more cysteine residues are present, one or more disulfide bonds may form between cysteine residues.

In an embodiment of the invention wherein one or more cysteine residues have been incorporated into the sequence of CRF, the polyethylene glycol moiety may be covalently bound to CRF through one or more of the cysteine residues. Alternatively, the polyethylene glycol moiety may be covalently bound through one or more of the existing 41 amino acids of CRF, including, but not limited to lysine, histidine, arginine, aspartic acid, glutamic acid, serine, as well as the N-terminus or C-terminus of the CRF polypeptide. In a particular embodiment of the invention, the CRF conjugate may have polyethylene glycol moieties attached via one or more lysine residues. The CRF conjugates of the invention include CRF which has been modified to include one or more polyethylene glycol polymers through a multitude of different sites in the CRF sequence. In one embodiment, the CRF conjugate comprises two PEG moieties bound to two cysteine residues. In one embodiment, the CRF-PEG conjugate comprises one or more PEG groups simultaneously bound to two cysteine residues that form a disulfide bond in a cysteine added variant of CRF. These conjugates may be produced via reductive cleavage of a disulfide bond, followed by a reaction in which the PEG moiety becomes bound to both thio groups. The resulting CRF conjugate contains a PEG moiety that bridges two sulfurs that had formed a disulfide bond. In a specific embodiment, the CRF conjugate contains a PEG bound to both the C-terminal and N-terminal cysteine residues of a cysteine added variant of CRF.

In a specific embodiment, a polyethylene glycol polymer is conjugated to a cysteine added variant of CRF according to general formula I:

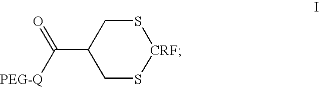

wherein both —S— are from cysteine residues that form a disulfide bond in a cysteine added variant of CRF, wherein Q represents a linking group which can be a direct bond, an alkylene group (preferably a C₁₋₁₀ alkylene group), or an optionally-substituted aryl or heteroaryl group;
wherein the aryl groups include phenyl, benzoyl and naphthyl groups;
wherein suitable heteroaryl groups include pyridine, pyrrole, furan, pyran, imidazole, pyrazole, oxazole, pyridazine, primidine and purine;
wherein linkage to the polymer may be by way of a hydrolytically labile bond, or by a non-labile bond.

Substituents which may be present on an optionally substituted aryl or heteroaryl group include for example one or more of the same or different substituents selected from —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺H₃, —N⁺HR₂, —N⁺H₂R, halogen, for example fluorine or chlorine, —C≡CR, —C═CR₂ and ¹³C═CHR, in which each R or R′ independently represents a hydrogen atom or an alkyl (preferably C₁₋₆) or an aryl (preferably phenyl) group. The presence of electron withdrawing substituents is especially preferred.

In one embodiment of formula I, PEG is conjugated to CRF according to formula II:

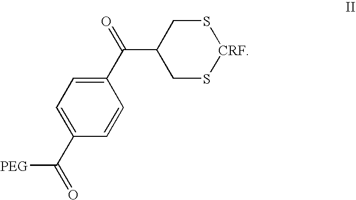

Two cysteine added variants of CRF may be bound together via a disulfide bond, to form a CRF dimer. The CRF dimer may be conjugated to a polyethylene glycol containing moiety. In one embodiment, the CRF dimer conjugate is bound to PEG through the disulfide bond that binds the two CRF polypeptides together.

In a specific embodiment, a polyethylene glycol polymer is conjugated to two cysteine added variants of CRF according to general formula III:

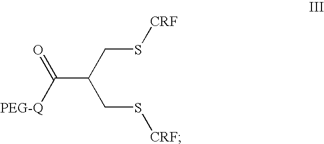

wherein both —S— are from cysteine residues that form a disulfide bond in a cysteine added variant of CRF, wherein Q represents a linking group which can be a direct bond, an alkylene group (preferably a C₁₋₁₀ alkylene group), or an optionally-substituted aryl or heteroaryl group;
wherein the aryl groups include phenyl, benzoyl and naphthyl groups;
wherein suitable heteroaryl groups include pyridine, pyrrole, furan, pyran, imidazole, pyrazole, oxazole, pyridazine, primidine and purine;
wherein linkage to the polymer may be by way of a hydrolytically labile bond, or by a non-labile bond.

Substituents which may be present on an optionally substituted aryl or heteroaryl group include for example one or more of the same or different substituents selected from —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺H₃, —N⁺HR₂, —N⁺H₂R, halogen, for example fluorine or chlorine, —C≡CR, —C═CR₂ and ¹³C═CHR, in which each R or R′ independently represents a hydrogen atom or an alkyl (preferably C₁₋₆) or an aryl (preferably phenyl) group. The presence of electron withdrawing substituents is especially preferred.

In one embodiment of formula III, PEG is conjugated to CRF according to formula IV:

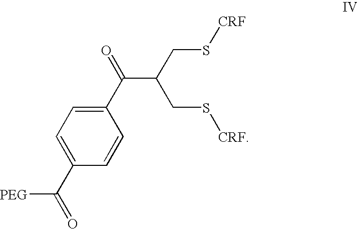

The CRF conjugates of the invention have one or more of the biological activities of unmodified CRF. Such biological activities include, for example, the ability to stimulate the release of ACTH, the ability to inhibit edema in vivo and the ability to bind to CRF receptors. The biological activity of CRF conjugates may be determined using the assays described herein.

Compared to unmodified CRF (i.e., CRF without a PEG attached), the conjugates of the present invention have an increased circulating half-life and plasma residence time and/or decreased clearance. In an embodiment of the invention, the CRF conjugates have increased clinical activity in vivo as compared to unmodified CRF. The conjugates of the invention have improved potency, stability, area under the curve and circulating half-life. The CRF conjugates of the invention have an improved pharmacokinetic profile as compared to unmodified CRF. The CRF conjugates of the invention may show an improvement in one or more parameters of the pharmacokinetic profile, including AUC, Cₘₐₓ, clearance (CL), half-life, and bioavailability as compared to unmodified CRF.

In accordance with the present invention, the CRF conjugates are useful for treating brain edema in patients in need thereof. In accordance with the present invention, such brain edema may be the result of injury or disease to the brain. In particular, the present invention relates to methods of treating brain edema resulting from primary or metastatic brain tumors comprising administering CRF conjugates to patients in need thereof.

The CRF conjugates of the invention are useful in treating patients by reducing inflammation and edema in those patients comprising administering a therapeutically effective amount of the novel CRF conjugates and formulations of the invention. The CRF conjugates of the invention are useful in providing vasoprotective effects which may be evidenced as a reduction in edema when administered to patients in need thereof. In particular, the methods of administering the CRF conjugates of the invention may be useful in reducing peritumoral brain edema. The administration of CRF conjugates to a patient for the treatment of brain edema may be combined with other therapeutics for the treatment of edema. In particular, the CRF conjugates of the invention may be used in combination with steroidal therapeutics for the treatment of brain edema, including, but not limited to glucocorticoids. Glucocorticoid steroids include hydrocortisone, cortisone acetate, prednisone, prednisolone, methylprednisone, dexamethasone, betamethasone, triamcinolone, beclomethasone, fludrocortisone acetate, alderstone and deoxycorticosterone acetate. In accordance with the invention, when CRF conjugates are administered in combination with other therapeutics for the treatment of brain edema, the other therapeutic may be administered concurrently, prior to or subsequently to the administration of the CRF conjugate.

In another aspect of the invention, the CRF conjugates may be administered to patients for the treatment of brain edema, wherein the conjugate is administered in a treatment regimen as a steroid sparing agent facilitating steroid taper. The invention also encompasses, a method for managing brain edema in a patient in need thereof comprising administering to the patient a therapeutically effective amount of a CRF conjugate and a steroid, wherein said method provides a steroid sparing effect. The present invention further provides a method for providing replacement therapy for steroid therapy in a subject receiving such therapy, said method comprising administration of a steroid-sparing amount of a CRF conjugate. The invention also provides a method for treating brain edema comprising a treatment regimen steroid in combination with a CRF conjugate, whereby total exposure to the steroid is reduced by the administration of the CRF conjugate.

The present invention relates to pharmaceutical compositions containing a CRF conjugate as the active ingredient. The CRF conjugate may be formulated with a pharmaceutically acceptable carrier. Due to the increased half-life of the CRF conjugate, the pharmaceutical compositions may contain a lower dose of CRF than typically administered to effectively treat edema. The pharmaceutical formulations of the invention may be formulated for parenteral administration, including, but not limited to, intradermal, subcutaneous, and intramuscular injections, and intravenous or intraosseous infusions. The pharmaceutical formulations of the present invention can take the form of solutions, suspensions, emulsions that include a CRF conjugate, such as CRF chemically modified with polyethylene glycol, and a pharmaceutically acceptable diluent, adjuvant or carrier, depending on the route of administration.

The pharmaceutical compositions of the invention are formulated to deliver a therapeutic dose of the CRF conjugate of the invention. The dose of the CRF conjugates contained in pharmaceutical formulation can range from 1 μg to 10 mg. In certain embodiments the dose of the CRF conjugate can range from 0.1 mg to 5 mg, or 0.3 mg to 2 mg. In certain embodiments, the dose of the CRF conjugate can be about 0.3 mg, about 0.5 mg, about 1 mg, about 2 mg, about 4 mg or about 5 mg.

The conjugates of the invention can be used in the same manner as unmodified CRF. However because of the improved properties of the CRF conjugates, the pharmaceutical formulations of the invention can be administered less frequently than the unmodified CRF. For example, the CRF conjugates may be administered once weekly instead of the once daily for unmodified CRF. The present invention also encompasses dosing regimens wherein the CRF derivatives may be administered once a day, once every two, three or four days, or once a week to effectively treat edema. Decreased frequency of administration is expected to result in improved patient compliance leading to improved treatment outcomes, as well as improved patient quality of life.

### 4. BRIEF DESCRIPTION OF THE DRAWINGS

FIG. 1 shows the amino acid sequences of the human and rat CRF peptides as compared to that of the ovine CRF peptide. Amino acids are presented as their standard one-letter designations. Amino acids in the ovine sequence which are presented in bold font and are underlined are those that differ from the human/rat CRF sequence.

### 5. DETAILED DESCRIPTION OF THE INVENTION

The present invention is based on conjugates of CRF that have been modified to include a moiety that results in a form of CRF that has an increased circulating half-life or plasma residence time as compared to unmodified CRF. The present invention is also related to methods of preparing such conjugates. The present invention further relates to methods of using such conjugates for reducing inflammation and edema in patients.

The CRF conjugates of the present invention have an improved pharmacokinetic profile as compared to unmodified CRF. The CRF conjugates of the invention may show an improvement in one or more parameters of the pharmacokinetic profile, including AUC, Cₘₐₓ, clearance (CL), half-life, and bioavailability as compared to unmodified CRF.

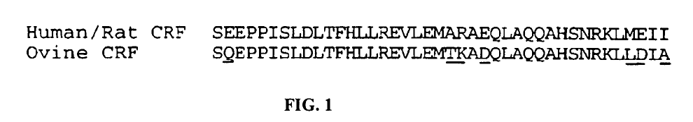

The CRF conjugates of the present invention include CRF with an unmodified amino acid sequence as is shown in FIG. 1, wherein one or more residues are covalently bound to polyethylene glycol. CRF conjugates of the present invention also include cysteine added variants of CRF, where one or more cysteine residues have been inserted into one of the CRF amino acid sequences shown in FIG. 1, or substituted for one or more residues of one of the CRF sequences shown in FIG. 1. The conjugated cysteine added variants of CRF, include CRF sequences with cysteine residues added at the N-terminus, the C-terminus, or both the N-terminus and C-terminus of one of the amino acid sequences shown in FIG. 1. When two or more cysteine residues are added to the sequence, two cysteine residues may together form a disulfide bond. In a specific embodiment, a cysteine residue at the C-terminus of the CRF sequence forms a disulfide bond with a cysteine residue at the N-terminus.

The CRF conjugates of the present invention can be used to treat edema by administering to a patient in need thereof a therapeutically acceptable amount of a CRF conjugate.

Another aspect of the invention is a method of treating edema comprising administering to a patient in need thereof a pharmaceutical composition comprising CRF chemically modified with polyethylene glycol and a pharmaceutically acceptable diluent, adjuvant or carrier.

Another aspect of the invention is a method for treating brain edema comprising administering CRF conjugate wherein the conjugate is administered in a treatment regimen as a steroid sparing agent facilitating steroid taper.

Another aspect of the invention is a method for managing brain edema in a patient in need thereof comprising administering to the patient a therapeutically effective amount of a CRF conjugate and a steroid, wherein said method provides a steroid sparing effect.

Another aspect of the invention is a method for providing replacement therapy for steroid therapy in a subject receiving such therapy, said method comprising administration of a steroid-sparing amount of CRF conjugate.

Another aspect of the invention is a method for treating brain edema comprising a treatment regimen steroid in combination with a CRF conjugate, whereby total exposure to the steroid is reduced by the administration of the CRF conjugate.

The “area under the curve” or “AUC”, as used herein in the context of administering a peptide drug to a patient, is defined as total area under the curve that describes the concentration of drug in systemic circulation in the patient as a function of time from zero to infinity.

As used herein the term “clearance” or “renal clearance” is defined as the volume of plasma that contains the amount of drug excreted per minute.

As used herein, the terms “corticotropin releasing factor”, “CRF”, “corticotrop(h)in-releasing hormone”, “CRH”, “corticoliberin”, “corticorelin”, “CRF-41” or grammatical equivalents thereof have a functional definition and refer to peptides which share one or more of the biological activities of the native, intact CRF peptide. Such biological activities include, for example, the ability to stimulate the release of ACTH, the ability to inhibit edema in vivo and the ability to bind to CRF receptors. Each of the above terms is intended to denote the 41 amino acid human, rat, ovine, sheep, goat, porcine and fish corticotropin releasing factor peptides and CRF peptides from other mammals, whether isolated from natural source extraction and purification, from recombinant cell culture systems or synthesized using peptide synthesis technology. These terms are also intended to denote other CRF-related peptides which share one or more of the biological activities of the native CRF peptides such as urocortin (Vaughan, J., et al., Nature 378:287-292 (1995), Donaldson, C. J., et al., Endocrinology 137(5):2167-2170 (1996) and Turnbull, A. V., et al., Eur. J. Pharm. 303:213-216 (1996)), urotensin I (Lederis, K., et al., Science 218:162-164 (1982)) and sauvagine (Montecucchi, P. C., et al., Int. J. Pep. Prot. Res. 16:191-199 (1980)).

The CRF peptides employed in the formulations of the present invention are preferably synthesized using solid- or solution-phase peptide synthesis techniques, however, other sources of the CRF peptide are readily available to the ordinarily skilled artisan. The amino acid sequences of the human, rat and ovine CRF peptides are presented in FIG. 1. The terms “corticotropin releasing factor” and “CRF” likewise cover biologically active CRF equivalents; e.g., peptides differing in one or more amino acids in the overall amino acid sequence as well as substitutional, deletional, insertional and modified amino acid variants of CRF which substantially retain the biological activity normally associated with the intact CRF peptide.

As used herein, the term “CRF conjugate” refers to a CRF polypeptide that has been modified to include a moiety that results in an improved pharmacokinetic profile as compared to unmodified CRF. The improvement in the pharmacokinetic profile may be observed as an improvement in one or more of the following parameters: potency, stability, area under the curve and circulating half-life.

As used herein, the term “cysteine added variant of CRF” refers to CRF that has been modified by the insertion of one or more cysteine residues into the unmodified CRF sequence shown in FIG. 1, or the substitution of one or more of the amino acid residues in the CRF polypeptide sequence shown in FIG. 1, for cysteine residues.

As used herein the term “half-life” or “t_{1/2},” in the context of administering a peptide drug to a patient, is defined as the time required for plasma concentration of a drug in a patient to be reduced by one half. There may be more than one half-life associated with the peptide drug depending on multiple clearance mechanisms, redistribution, and other mechanisms well known in the art. Usually, alpha and beta half-lives are defined such that the alpha phase is associated with redistribution, and the beta phase is associated with clearance. However, with protein drugs that are, for the most part, confined to the bloodstream, there can be at least two clearance half-lives. The precise impact of PEGylation on alpha phase and beta phase half-lives will vary depending upon the size and other parameters, as is well known in the art. Further explanation of “half-life” is found in Pharmaceutical Biotechnology (1997, DFA Crommelin and R D Sindelar, eds., Harwood Publishers, Amsterdam, pp 101 120).

As used herein, when referring to the administration of CRF conjugates of the invention, the term a “patient in need thereof,” refers to a patient who has been diagnosed with a condition that may be treated by CRF, e.g., brain edema.

As used herein, the term “pharmaceutically acceptable” when used in reference to the formulations of the present invention denotes that a formulation does not result in an unacceptable level of irritation in the subject to whom the formulation is administered by any known administration regimen. What constitutes an unacceptable level of irritation will be readily determinable by those of ordinary skill in the art and will take into account erythema and eschar formation as well as the degree of edema associated with administration of the formulation.

As used herein the term “residence time,” in the context of administering a peptide drug to a patient, is defined as the average time that drug stays in the body of the patient after dosing.

As used herein, the terms “treat”, “treating” or “treatment of” mean that the severity of a subject's condition is reduced or at least partially improved or ameliorated and/or that some alleviation, mitigation or decrease in at least one clinical symptom is achieved and/or there is an inhibition or delay in the progression of the condition and/or delay in the progression of the onset of disease or illness. The terms “treat”, “treating” or “treatment of” also means managing the disease state, e.g., brain edema.

As used herein, a “sufficient amount” or an “amount sufficient to” achieve a particular result refers to an amount of CRF conjugate that is effective to produce a desired effect, which is optionally a therapeutic effect (i.e., by administration of a therapeutically effective amount). For example, a “sufficient amount” or “an amount sufficient to” can be an amount that is effective to reduce the amount of steroid required to manage the edema.

As used herein, a “therapeutically effective” amount is an amount that provides some improvement or benefit to the subject. Alternatively stated, a “therapeutically effective” amount is an amount that provides some alleviation, mitigation, and/or decrease in at least one clinical symptom. Clinical symptoms associated with the disorder that can be treated by the methods of the invention are well-known to those skilled in the art. Further, those skilled in the art will appreciate that the therapeutic effects need not be complete or curative, as long as some benefit is provided to the subject.

### 5.1 CRF Conjugates

The CRF conjugates of the invention have one or more of the biological activities of unmodified CRF. Such biological activities include, for example, the ability to stimulate the release of ACTH, the ability to inhibit edema in vivo and the ability to bind to CRF receptors. The biological activity of CRF conjugates may be determined using the assays described herein.

Compared to unmodified CRF (i.e., CRF without a PEG attached), the conjugates of the present invention have an increased circulating half-life and plasma residence time and/or decreased clearance. In an embodiment of the invention, the CRF conjugates have increased clinical activity in vivo as compared to unmodified CRF. The conjugates of the invention have improved potency, stability, area under the curve and circulating half-life. The CRF conjugates of the invention have an improved pharmacokinetic profile as compared to unmodified CRF. The CRF conjugates of the invention may show an improvement in one or more parameters of the pharmacokinetic profile, including AUC, Cₘₐₓ, clearance (CL), half-life, and bioavailability as compared to unmodified CRF.

CRF to be modified in accordance with the invention may be obtained and isolated from natural sources. CRF to be modified in accordance with the invention may be expressed recombinantly. CRF to be modified in accordance with the invention may be synthetically produced.

In one embodiment, the CRF component of the CRF conjugate has the sequence identified as human CRF identified in FIG. 1. In one embodiment, the CRF component of the CRF conjugate has the sequence identified as rat or ovine CRF identified in FIG. 1. Alternatively, the sequence of CRF may be modified or derivatized to include one or more changes in the amino acid sequence, including, but not limited to insertions, deletions or substitutions. In yet another embodiment the sequence of CRF has been modified to include one or more cysteine residues. The sequence of CRF may include cysteine as a substitution of one or more of the existing residues of CRF, alternatively, the cysteine residue may be incorporated as an addition to the existing sequence of CRF. The cysteine residues may be inserted within the sequence of CRF, the cysteine residue may be added to the amino or carboxy terminus of the sequence, or a cysteine residue may be added at both the amino and carboxy termini.

A CRF-PEG conjugate containing a PEG bound to one or more functional groups of the naturally occurring CRF polypeptide leads to increased circulating half-life and plasma residence time, decreased clearance, and increased clinical activity in vivo. CRF may be modified by covalently binding a polyethylene glycol polymer through one or more of its 41-amino acids including, but not limited to lysine, histidine, arginine, aspartic acid, glutamic acid, serine, as well as the N-terminal α-amino and C-terminal carboxylate groups of the protein. Polyethylene glycol polymer units can be linear or branched. The CRF-PEG conjugate may be delivered intravenously or subcutaneously via injection.

One aspect of the invention is a CRF-PEG conjugate, wherein PEG is bound to one or more amino groups of CRF. Another aspect of the invention is a CRF-PEG conjugate, wherein a polyethylene glycol polymer is bound to one or more carboxyl groups of CRF. Another aspect of the invention is a CRF-PEG conjugate where a polyethylene glycol polymer is bound to one or more alcohol groups of CRF.

Another aspect of the invention is a CRF-PEG conjugate where a polyethylene glycol polymer is bound to the lysine residue. The ε-amino group of lysine in CRF can be readily PEGylated by a variety of techniques, including but not limited to alkylation and acylation.

Another aspect of the invention is a CRF conjugate where a polyethylene glycol polymer is bound to the N-terminal α-amino group. The N-terminal α-amino residue of CRF can form a PEG conjugate by a variety of techniques including, but not limited to alkylation or acylation of the N-terminal α-amino group.

Another aspect of the invention are cysteine added variants of CRF that contain one or more PEG conjugated cysteine residues that have been substituted for naturally occurring residues in the CRF polypeptide sequence. Cysteine substituted CRF can be produced recombinantly by expressing DNA with point mutations that result in the substitution of a cysteine for a residue in naturally occurring CRF. For example the codon TCT, which codes for serine, can be mutated to TGC, which codes for cysteine, so in place of one of the serine residues a cysteine will be expressed. If CRF is produced via synthetic means, in the course of the synthesis it is possible to substitute a cysteine residue in place of one or more residues that naturally occur in CRF. The cysteine can then be selectively conjugated to a polyethylene glycol polymer.

Another aspect of the invention are cysteine added variants of CRF that contain one or more PEG conjugated cysteine residues that have been inserted into the naturally occurring CRF sequence shown in FIG. 1. If CRF is produced recombinantly, this can be done by inserting one or more cysteine codon(s) into the DNA sequence that codes for CRF. In solid phase protein synthesis, cysteines are added at any point of the protein synthesis by introducing an additional cysteine residue where desired. The cysteine can then be selectively bound to a polyethylene glycol polymer.

Another aspect of the invention is a cysteine added variant of CRF that contains a PEG conjugated cysteine residue inserted at the N-terminus. Another aspect of the invention is a CRF conjugate that contains a PEG bound to a cysteine residue inserted at the C-terminus. Another aspect of the invention is a CRF conjugate that contains PEG bound to cysteine residues inserted at both the N-terminus and the C-terminus. In a specific embodiment, a cysteine residue at the C-terminus of the CRF sequence forms a disulfide bond with a cysteine residue at the N-terminus. In one embodiment, the CRF-PEG conjugate comprises one or more PEG groups simultaneously bound to two cysteine residues that form a disulfide bond in a cysteine added variant of CRF. These conjugates may be produced via reductive cleavage of a disulfide bond, followed by a reaction in which the PEG moiety becomes bound to both thio groups. The resulting CRF conjugate contains a PEG moiety that bridges two sulfurs that had formed a disulfide bond. In a specific embodiment, the CRF conjugate contains a PEG bound to both the C-terminal and N-terminal cysteine residue of a cysteine added variant of CRF.

In a specific embodiment, a polyethylene glycol polymer is conjugated to a cysteine added variant of CRF according to general formula I:

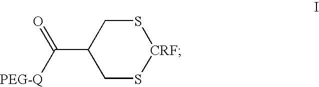

wherein both —S— are from cysteine residues that form a disulfide bond in a cysteine added variant of CRF, wherein Q represents a linking group which can be a direct bond, an alkylene group (preferably a C₁₋₁₀ alkylene group), or an optionally-substituted aryl or heteroaryl group;
wherein the aryl groups include phenyl, benzoyl and naphthyl groups;
wherein suitable heteroaryl groups include pyridine, pyrrole, furan, pyran, imidazole, pyrazole, oxazole, pyridazine, primidine and purine;
wherein linkage to the polymer may be by way of a hydrolytically labile bond, or by a non-labile bond.

Substituents which may be present on an optionally substituted aryl or heteroaryl group include for example one or more of the same or different substituents selected from —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺H₃, —N⁺HR₂, —N⁺H₂R, halogen, for example fluorine or chlorine, —C═CR, —C═CR₂ and ¹³C═CHR, in which each R or R′ independently represents a hydrogen atom or an alkyl (preferably C₁₋₆) or an aryl (preferably phenyl) group. The presence of electron withdrawing substituents is especially preferred.

In one embodiment of formula I, PEG is conjugated to CRF according to formula II:

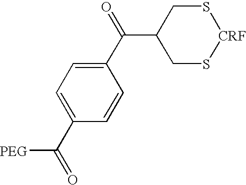

There are several different types of polyethylene glycol polymers that will form conjugates with CRF polypeptides. There are linear PEG polymers that contain a single polyethylene glycol chain, and there are branched or multi-arm PEG polymers. Branched polyethylene glycol contains 2 or more separate linear PEG chains bound together through a unifying group. For example, two PEG polymers may be bound together by a lysine residue. One linear PEG chain is bound to the α-amino group, while the other PEG chain is bound to the ε-amino group. The remaining carboxyl group of the lysine core is left available for covalent attachment to a protein. Both linear and branched polyethylene glycol polymers are commercially available in a range of molecular weights.

In one aspect of the invention, a CRF-PEG conjugate contains one or more linear polyethylene glycol polymers bound to CRF, wherein each PEG having a molecular weight between about 2 kDa to about 100 KDa. In another aspect of the invention, a CRF-PEG conjugate contains one or more linear polyethylene glycol polymers bound to CRF, wherein each branched PEG has a molecular weight between about 5 kDa to about 40 kDa.

A CRF-PEG conjugate of this invention may contain one or more branched polyethylene glycol polymers bound to CRF, wherein each branched PEG has a molecular weight between about 2 kDa to about 100 kDa. In another aspect of the invention, a CRF-PEG conjugate contains one or more branched polyethylene glycol polymers bound to CRF, wherein each branched PEG has a molecular weight between about 5 kDa to about 40 kDa.

### 5.2 Methods of Producing CRF Derivatives

### 5.2.1. PEGylation of Amino Groups

CRF can be conjugated with polyethylene glycol, without the modification of the original 41 residue polypeptide chains. Both the lysine ε-amino group and the N-terminal α-amino group can be PEGylated by alkylation and acylation as demonstrated below.

The ε-amino group of lysine is a commonly used group for PEG conjugation of proteins, and CRF contains a single lysine residue. The PEG conjugation of lysine via its ε-amino group may be accomplished by methods including, but not limited to acylation and alkylation. When a PEG-aldehyde reacts with an amino group a Schiff base is formed. Harris and Herati (U.S. Pat. No. 5,252,714) incorporated herein by reference in its entirety, use polyethylene glycol propionaldehyde as the PEG aldehyde. The Schiff base is then reduced by sodium cyanoborohydride to produce a CRF-PEG conjugate. A drawback to this method is that Schiff base formation is slow, often requiring a day or more to occur. An alternative alkylation strategy is the use of PEG-tresyl chloride as the PEG alkylating reagent. The advantage of PEG-tresyl chloride is that it shows enhanced reactivity towards amino groups as demonstrated in Delgado (U.S. Pat. No. 5,349,052) incorporated herein by reference in its entirety. PEG conjugates of CRF can be further purified and isolated by techniques known in the art.

PEG conjugation of the ε-amino group of lysine via acylation is a technique known in the art for conjugating PEG polymers to the ε-amino group of lysine residues, such as the lysine residue in CRF. Commonly employed PEG reagents are N-hydroxysuccinimidyl (NHS) esters of PEG as shown by Veronese, F. M. Biomaterials. 22(2001): 405-417. Other PEG acylation reagents are PEG-p-nitophenylcarbonate and PEG-trichlorophenylcarbonate in Veronese F. M. et. al. Appl. Biochem. Biotechnol 11(1985): 141-152, PEG oxycarbonylimidizole in Beauchamp, C. O. et al. Anal. Biochem. 131(1983): 25-33, and PEG-benzotriazole carbonate in Dolence et. al. (U.S. Pat. No. 5,650,234) incorporated herein by reference in its entirety,. CRF-PEG conjugates synthesized by acylation can be purified and isolated by methods known in the art, including gel filtration or size exclusion chromatography.

The N-terminal α-amino group can be selectively bound to polyethylene glycol polymers, as taught in Kinstler (U.S. Pat. No. 6,586,398) incorporated herein by reference in its entirety.

One method of N-terminal PEGylation, is reductive alkylation with a PEG aldehyde, in a procedure similar to that described earlier. For example, a large excess methoxy PEG aldehyde can be mixed with the CRF protein in a buffered solution of pH 4-6. Sodium cyanoborohydride is added to the mixture, and the desired CRF-PEG conjugates can be isolated and purified by methods known in the art. The N-terminal amino group can also be modified by acylation with an activated NHS ester of PEG. To a slightly basic buffered solution of CRF, can be added a large excess of the PEG ester of NHS. After the reaction is complete, the CRF-PEG conjugate can be isolated and purified by methods known in the art.

### 5.2.2. Insertion and Substitution of Cysteine Residues

CRF derivatives where cysteines have been inserted or substituted can be produced by recombinant means using techniques known in the art. Expression of the desired cysteine substituted or inserted derivative may be done in either eukaryotic or bacterial cells by methods used by Shaw (U.S. Pat. No. 5,166,322) incorporated herein by reference in its entirety, for IL-3 cysteine added variants. Modifications to the naturally occurring CRF protein can be accomplished site directed PCR-based mutagenesis. Cox III (U.S. Pat. No. 7,214,779) incorporated herein by reference in its entirety, discloses cysteine added variants of granulocyte-macrophage colony stimulating factor (GCSF) that are produced by recombinant means. Cysteine added variants of CRF can also be made by synthetic methods. Cysteine residues can be substituted for another amino acid residue during the course of the synthesis. By adding an additional step to the solid phase synthesis of CRF, a cysteine residue can also be inserted where desired in the polypeptide sequence. In solid phase synthesis, the cysteine may be added to the C-terminus of the CRF sequence at the first step of the synthesis. Alternatively, the cysteine may be added to the N-terminus of the CRF sequence, at the last step of solid phase synthesis. By adding a cysteine residue at the first and last steps of the solid phase synthesis, cysteine residues would be present at the C-terminus and N-terminus of the resulting cysteine added variant of CRF. A disulfide bond between the two cysteines may further result.

### 5.2.3. Techniques for the PEGylation of Cystine Residues

A number of methods exist in the art for forming polyethylene glycol conjugated, or PEGylated cysteine residues. The advantage of these techniques are that they are selective for cysteine, which means that other amino acid residue side chains remain untouched by these methods. In scheme 1a, the activated disulphide, PEG ortho-pyridyl-disulphide, reacts with thiols to form the more stable symmetric disulphide. In scheme 1b, a cysteine residue reacts with PEG-maleamide, via a thiol addition to the activated double bond in a Michael addition reaction. In scheme 1c a conjugate attack by the thiol on the activated terminal vinyl group of PEG-vinylsulphone, yields the PEGylated cysteine residue. In scheme 1d the cysteine thiol displaces the iodide via a nucleophilic attack to yield the PEG conjugated cysteine residue.

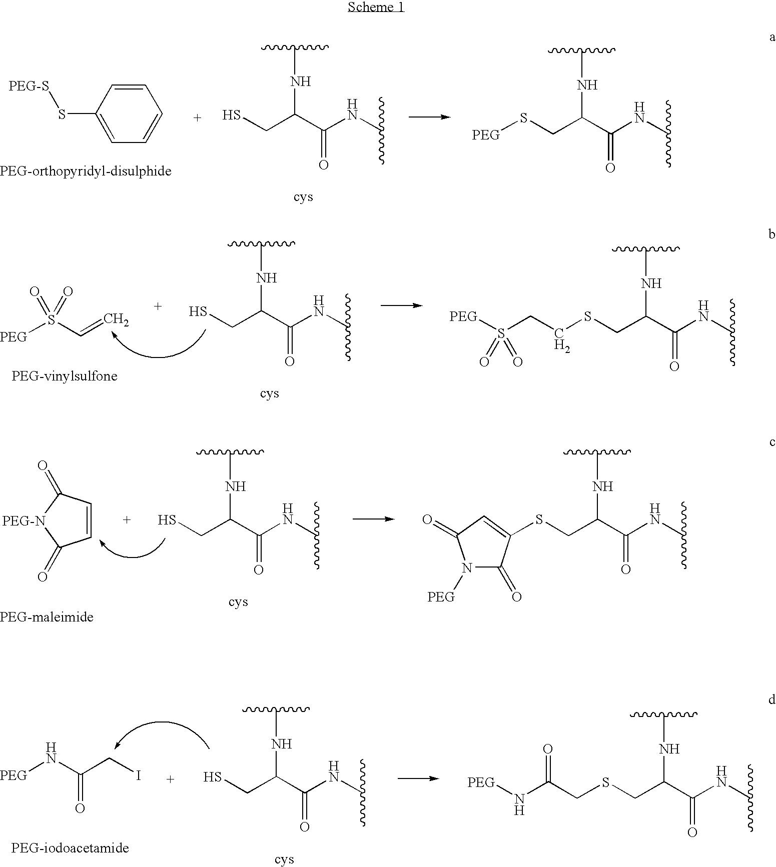

Two cysteine groups that together form a disulfide bond may also be PEGylated selectively by using the technique shown in scheme 2. The native disulfide bond is first reduced. One of the resulting thiols from this bond can nucleophilicly attack an electrophilic group, such as a 1,4-addition to an enone. This is followed by the departure of a leaving group, such as, e.g. a sulfone. The subsequent elimination to a second enone, followed by 1,4-addition by the remaining thiol leads to the bridged disulfide with a PEG group attached.

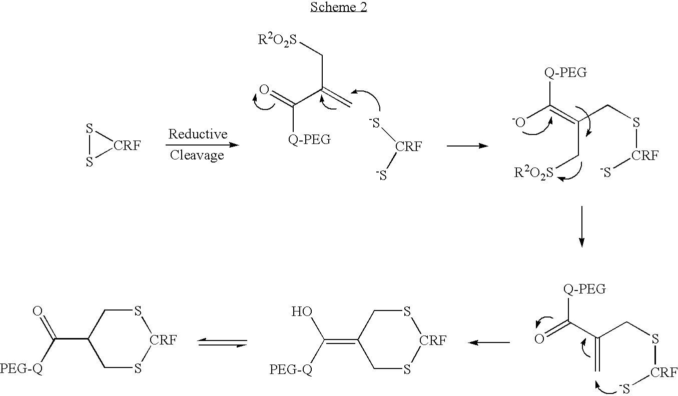

For dimers of cysteine added variants of CRF, the PEGylation reaction proceeds via the scheme 2b.

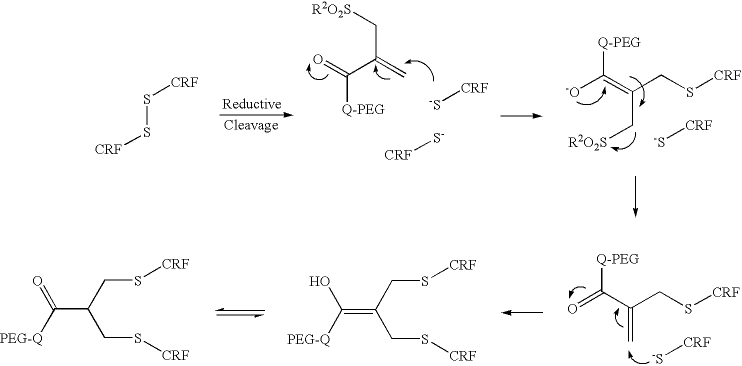

### 5.3 Methods of Assaying Biological Activity

The CRF conjugates of the invention have one or more of the biological activities of unmodified CRF. Such biological activities include, for example, the ability to stimulate the release of ACTH, the ability to inhibit edema in vivo and the ability to bind to CRF receptors. The biological activity of CRF conjugates may be determined using biological assays known in the art, or the assay described in section 6.3.

### 5.4 Methods of Treating Edema

The present invention is also directed to methods of treating edema. The methods described herein include methods of treating edema comprising administering to a patient in need thereof a pharmaceutical composition comprising a CRF conjugate. In certain embodiments the CRF conjugate is CRF chemically modified with polyethylene glycol.

The present invention is also directed to methods of treating brain edema comprising administering CRF conjugate, wherein the conjugate is administered in a treatment regimen as a steroid sparing agent facilitating steroid taper.

In certain embodiments the methods described herein include methods for managing brain edema in a patient in need thereof comprising administering to the patient a therapeutically effective amount of a CRF conjugate and a steroid, wherein said method provides a steroid sparing effect. The CRF conjugates described here can be co-administered with any steroid including glucocorticoids, which are a class of steroid hormones characterized by an ability to bind with the cortisol receptor. Glucocorticoids steroids include hydrocortisone, cortisone acetate, prednisone, prednisolone, methylprednisolone, dexamethasone, betamethasone, triamcinolone, beclometasone, fludrocortisone acetate, aldosterone and deoxycorticosterone acetate.

In other embodiments, the methods described herein include methods for treating brain edema comprising a treatment regimen comprising administering to in a patient in need thereof a steroid in combination with a CRF conjugate, whereby total exposure to the steroid is reduced by the administration of the CRF conjugate.

The present invention also includes methods for providing replacement therapy for steroid therapy in a subject receiving such therapy, said method comprising administration of a steroid-sparing amount of CRF conjugate.

The total daily dose of the CRF conjugates described herein, such as CRF chemically modified with polyethylene glycol, can range from 1 μg to 10 mg. In certain embodiments the total daily dose of CRF conjugate can be 0.1 mg to 5 mg, or 0.3 mg to 2 mg. For example, the total daily dose of CRF chemically modified with polyethylene glycol can be about 0.3 mg, about 0.5 mg, about 1 mg, about 2 mg, about 4 mg or about 5 mg. The CRF conjugate can be administered once a day or multiple times a day until the desired daily dose of the CRF conjugate is reached. For example, 0.5 mg or 1.0 mg of a CRF conjugate can be administered 4 time a day to achieve a total daily dose of 2 mg or 4 mg of the CRF conjugate.

Examples of routes of administration of the CRF conjugate include parenteral routes such as, but not limited to, intradermal, subcutaneous, and intramuscular injections, and intravenous or intraosseous infusions. The compositions of the present invention can take the form of solutions, suspensions, emulsions that include a CRF conjugate, such as CRF chemically modified with polyethylene glycol, and a pharmaceutically acceptable diluent, adjuvant or carrier, depending on the route of administration.

In certain embodiments the CRF conjugates described herein can be administered by subcutaneous injection in an amount of 0.1 μg/kg to 1000 μg/kg. CRF conjugates can be administered subcutaneously in an amount of 1 μg/kg to 500 μg/kg, or 2 μg/kg to 100 μg/kg, or 2 μg/kg to 80 μg/kg, or 4 μg/kg to 40 μg/kg, or 5 μg/kg to 20 μg/kg. For example, CRF conjugates can be administered in 10 μg/kg, 30 μg/kg, 60 μg/kg, 100 μg/kg and 300 μg/kg doses.

In other embodiments, the CRF conjugates described herein can be administered by subcutaneous injection in an amount of 1 μg to 100 mg. CRF conjugates can be administered subcutaneously in an amount of 1 μg to 80 mg, 10 μg to 50 mg, 100 μg to 40 mg, 300 μg to 10 mg, 600 μg to 1 mg, and 800 μg to 1 mg. For example, CRF conjugates can be administered subcutaneously in 100 μg, 300 μg, 600 μg, 1 mg, 2 mg, 4 mg and 5 mg doses.

The CRF conjugates administered subcutaneously can be administered once a day or multiple times a day. For example, the dosages of CRF conjugates administered subcutaneously can be administered every hour, every two hours, every three hours, every four hours, every six hours, every eight hours or every 12 hours. Alternatively, the CRF conjugates can be administered once every two, three, four, five or six days. In certain embodiments the CRF conjugates can be administered once a week, once every two, three or four weeks or once a month. Dosages of CRF conjugates that are administered once a week or longer can be administered in the form of a depot.

In still other embodiments the CRF conjugates can be administered by intravenous infusion in an amount of 0.1 μg/kg/h to 100 μg/kg/h. For example, CRF conjugates can be administered intravenously in an amount of 1 μg/kg/h to 100 μg/kg/h, or 2 μg/kg/h to 80 μg/kg/h, or 2 μg/kg/h to 50 μg/kg/h, or 4 μg/kg/h to 40 μg/kg/h, or 5 μg/kg/h to 20 μg/kg/h.

In other embodiments the CRF conjugates can be administered intravenously in an amount of 1 μg/kg to 1000 μg/kg. For example CRF conjugates can be administered intravenously in an amount of 1 μg/kg to 100 μg/kg, or 2 μg/kg to 80 μg/kg, or 2 μg/kg to 50 μg/kg, or 4 μg/kg to 40 μg/kg, or 5 μg/kg to 20 μg/kg. For example, CRF conjugates can be administered in 0.5 μg/kg to 1 μg/kg, or 2 μg/kg to 8 μg/kg, or 4 μg/kg to 8 μg/kg, or 5 μg/kg doses.

The CRF conjugates described herein can be administered intravenously over a period of an hour or less than an hour. In certain embodiments the CRF conjugates can be administered intravenously over a period of one hour or more. For example, the dosages of CRF chemically modified with polyethylene glycol administered intravenously, discussed above can be administered over a period of 10 min., 30 min., 45 min., one hour, two hours, four hours, eight hours, 12 hours, 24 hours, 48 hours or 72 hours.

### 5.4.1. Dosing Regimens

Dosing regimens include administration of the CRF conjugates of the invention every other day or once weekly to a patent suffering from edema resulting from disease or injury to the brain or nervous system.

### 5.4.2. Pharmaceutical Compositions

The present invention relates to pharmaceutical compositions containing a CRF conjugate as the active ingredient. The CRF conjugate may be formulated with a pharmaceutically acceptable carrier. Due to the increased half-life of the CRF conjugate, the pharmaceutical compositions may contain a lower dose of CRF than typically administered to effectively treat edema. The pharmaceutical formulations of the invention may be formulated for parenteral administration, including, but not limited to, intradermal, subcutaneous, and intramuscular injections, and intravenous or intraosseous infusions. The pharmaceutical formulations of the present invention can take the form of solutions, suspensions, emulsions that include a CRF conjugate, such as CRF chemically modified with polyethylene glycol, and a pharmaceutically acceptable diluent, adjuvant or carrier, depending on the route of administration.

The pharmaceutical compositions of the invention are formulated to deliver a therapeutic dose of the CRF conjugate of the invention. The dose of the CRF conjugates contained in pharmaceutical formulation can range from 1 μg to 10 mg. In certain embodiments the dose of the CRF conjugate can range from 0.1 mg to 5 mg, or 0.3 mg to 2 mg. In certain embodiments, the dose of the CRF conjugate can be about 0.3 mg, about 0.5 mg, about 1 mg, about 2 mg, about 4 mg or about 5 mg.

The present invention is also directed to methods of treating edema by administering to a patient in need thereof a CRF conjugate and an additional therapeutic agent. The additional therapeutic agent can be any agent that can alleviate edema or when in combination with the CRF conjugate, improve the conjugate's effect on edema or wherein the CRF conjugate can improve the effect of the additional therapeutic agent on the edema.

Suitable additional therapeutic agents include anti-inflammatory agents such as, but not limited to, corticosteroids. Corticosteroids include glucocorticoids and mineralocorticoids such as alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol or combinations thereof.

Suitable additional agents also include diuretics such as loop diuretics, osmotic diuretics proximal diuretics, distal convoluted tubule diuretics and cortical collecting tubule diuretics. For example, suitable diuretics include, but are not limited to, glucose, mannitol, bumetanide, ethacrynic acid, furosemide, torsemide, amiloride, spironolactone, triamterene, bendroflumethiazide, hydrochlorothiazide, acetazolamide, dorzolamide, Phosphodiesterase, chlorthalidone, caffeine, metolazone or a combination thereof.

Additional agents that can be co-administered with the CRF conjugate include anti-neoplastic, anti-proliferative, anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins.

Additionally, the CRF conjugates described herein can be co-administered with other anti-cancer treatments such as, radiotherapy, chemotherapy, photodynamic therapy, surgery or other immunotherapy.

The CRF conjugate and the additional therapeutic agent can be administered sequentially or simultaneously. If administered sequentially, the order of administration is flexible. For instance, the CRF conjugate can be administered prior to administration of the additional therapeutic agent. Alternatively, administration of the additional therapeutic agent can precede administration of the CRF conjugate.

Whether they are administered as separate compositions or in one composition, each composition is preferably pharmaceutically suitable for administration. Moreover, the CRF conjugate and the therapeutic agent, if administered separately, can be administered by the same or different modes of administration.

### 6. EXAMPLES

### 6.1 Syntheses of CRF Conjugates

The CRF conjugates of the invention can be readily synthesized using synthetic methods known in the art. The following synthetic examples demonstrate the syntheses of CRF-PEG conjugates, including CRF-PEG conjugates of cysteine added variants of CRF.

### 6.1.1. Example 1

### PEGylation of the CRF Lysine Residue

The alkylation of the ε-amino group of the lysine residue in hCRF can be accomplished via reductive alkylation using PEG-propionaldehyde as the PEGylation agent. Human-CRF (1mg) is stirred with an excess of PEG-propionaldehyde (3 mg) and a slight molar excess of sodium cyanoborohydride at room temperature in pH 9 borate buffer. High pH is used to avoid reduction of the aldehyde before Schiff base formation. In order to isolate the desired CRF-PEG conjugate, the mixture undergoes dialysis against phosphate buffered saline. In a system consisting of 8% dextran T-40, 6% PEG 8000, 0.15 M NaCl, and 0.010 M sodium phosphate pH 7.2, the CRF-PEG conjugate migrates to the top phase, while the unmodified CRF migrates to the bottom phase. The desired CRF-PEG conjugate may be further isolated by gel filtration chromatography.

Acylation of human-CRF with a polyethylene glycol group can be done using a PEG activated NHS ester. Human-CRF is solubilized (2-4 mg/ml) in 50 mM Bicine buffer. To the buffered hCRF solution is added to 10-20 molar equivalents of the PEG activated NHS ester. The reaction is stirred for 1 hour at room temperature. Upon completion of the reaction, the desired CRF-PEG conjugate may be isolated by gel filtration chromatography.

### 6.1.2. Example 2

### PEGylation of Cysteine Added Variants of CRF

As discussed in section 5.2.3 there are a number of reagents that can be employed to covalently bind a cysteine residue to polyethylene glycol. This example employs PEG-maleimide or maleimido-PEG as the PEGylation reagent. A cysteine added variant of CRF is diluted to 200 μg/ml in 20 mM Piperazine-1,4-bis(2-ethanesulfonic acid) (PIPES) pH 6.75 buffer, 0.6M NaCl, and 1% glycerol. Maleimido-PEG (1 μl) is dissolved in a 10 μl buffer composed of 20 mM Tris pH 7.4, 0.1M NaCl, and 0.01% Tween. The maleimdo-PEG may be diluted until the desired concentration is reached for reaction, and then it is added to the solution of CRF. Up to a 20-fold excess of maleimido-PEG may be used. The reaction is allowed to occur at room temperature for one hour, but the reaction may also occur at 4° C. with longer reaction times. Upon completion the resulting cysteine added variant CRF-PEG conjugate may be purified by gel filtration chromatography.

### 6.1.3. Example 3

### PEGylation of the cys-hCRF-cys via Disulfide Bond Bridging

To cys-hCRF-cys, which has cyclized via formation of a disulfide bond between the two cysteine residues, is added aqueous urea solution 2-mercaptoethanol. The pH of the resulting solution is adjusted to pH 8.5 using a 10% aqueous solution of methylamine. The reaction solution is then bubbled with nitrogen for approximately 30 min. Still purging with nitrogen the tube is heated at 37° C. The reaction mixture is then cooled in an ice-salt water bath and 10 mL of an argon purged chilled solution of 1N HCl:absolute ethanol is added to the reaction solution. A precipitation occurs and the precipitate is isolated by centrifugation and then washed three times with further portions of the HCl:absolute ethanol mixture and twice with nitrogen purged chilled diethyl ether. After each washing the precipitate is isolated by centrifugation. The washed precipitate is then dissolved in nitrogen purged deionized water and freeze-dried to afford a dry solid. Partial reduction of cys-hCRF-cys may be confirmed and quantitated using Ellman's Test, which gives the number free thiols per protein molecule.

In an eppendorf, the partially reduced cys-hCRF-cys is dissolved in argon purged pH 8 ammonia solution. In a separate eppendorf, the polymer conjugating reagent, α-methoxy-ω-4-[2,2-bis[(p-tolylsulfonyl)-methyl]acetyl]benzamide derived from poly(ethylene)glycol is also dissolved in ammonia solution and the resulting solution is added to the Factor IX solution. The PEG eppendorf is washed with fresh ammonia solution and this is also added to the main reaction eppendorf. The reaction eppendorf is then closed under argon and heated at 37° C. for approximately 24 h and then allowed to cool to room temperature. The cooled reaction solution is then analyzed by sodium dodecyl sulfate-polyacrylamide gel electrophoresis (SDS-PAGE).

### 6.1.4. Example 4

### Solid Phase Synthesis of a N-terminal Cysteine Added Variant of CRF

Synthesis of cysteine added variants of CRF can be made via solid phase peptide synthesis techniques. A cysteine residue may be inserted at the N-terminus of CRF at the last step of the synthesis as shown in scheme 3.

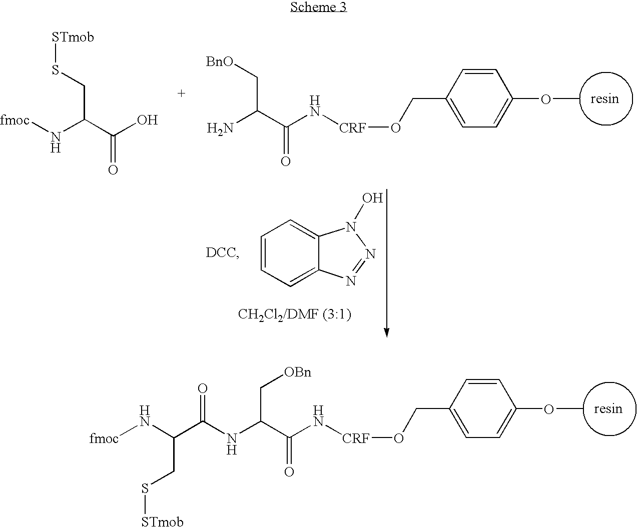

The N-terminus of unmodified hCRF is a serine residue, so it is to the α-amino group of serine that a cysteine residue is bound. A cysteine residue protected by S-2,4,6-trimethoxybenzyl (Tmob) is added to a solution of N-terminal deprotected CRF in a solution of dichloromethane/DMF in a ratio of 3:1. The coupling reaction can be monitored by the ninhydrin test for completion. Once complete, the solid phase is washed with dichloromethane and methanol, and an additional wash with DMF can be performed after this coupling step, to yield the solid phase coupled intermediate above.

In this example, cysteine is the last amino acid added. Once coupled, removal from the solid support is accomplished with anhydrous trifluroacetic acid, followed by universal deprotection of all of the protecting groups on side chains, yielding an N-terminal cysteine added variant of CRF. The final polypeptide can be isolated by gel filtration chromatography. Insertions and substitutions of additional cysteine residues may be accomplished by similar preparations in the desired locations of the CRF polypeptide.

### 6.1.5. Example 5

### Solid Phase Synthesis of a C-terminal Cysteine Added Variant of CRF

Synthesis of cysteine added variants of CRF can be made via solid phase peptide synthesis techniques. A cysteine residue may be inserted at the C-terminus of CRF at the first step of the synthesis as shown in scheme 4.

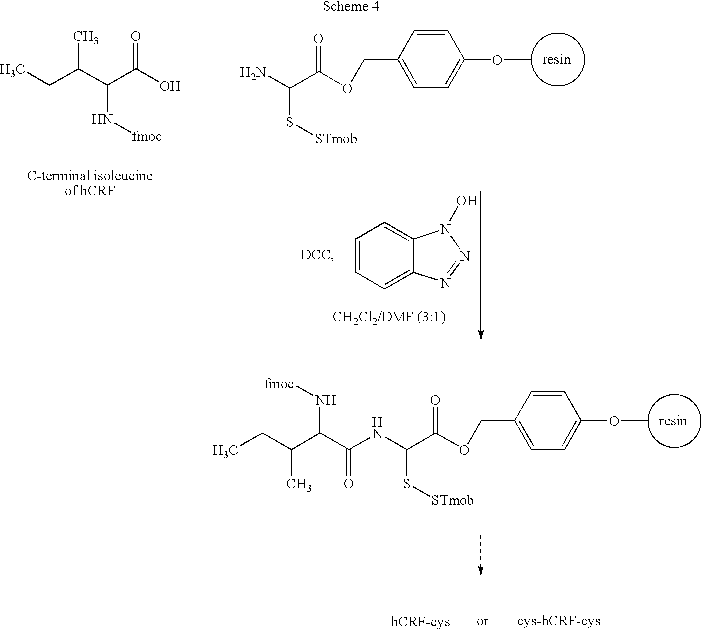

The C-terminus of unmodified hCRF is an isoleucine residue, so it is to the α-amino group of the C-terminal cysteine that an isoleucine residue is bound. A cysteine residue protected by S-2,4,6-trimethoxybenzyl (Tmob) is attached to the resin polymer. Isoleucine is added to the solution with DCC and 1H-benzo[d][1,2,3]triazol-1-ol in dichloromethane/DMF in a ratio of 3:1. The coupling reaction can be monitored by the ninhydrin test for completion. Once complete, the solid phase is washed with dichloromethane and methanol, and an additional wash with DMF can be performed after this coupling step, to yield the solid phase coupled intermediate above.

The sequential addition of amino acid residues is continued until the synthesis of the desired CRF variant is complete.

### 6.2 Human CRF Assay

The CRF conjugates of the invention have one or more of the biological activities of unmodified CRF. The biological activity of the CRF conjugates may be determined using the bioassays described herein. The CRF conjugates may have the same level of biologic activity as compared to unmodified CRF. Alternatively, the CRF conjugates may have lower levels of biologic activities when compared to unmodified CRF.

The following is a bioassay for CRF. The bioassay is to be based upon the binding of radio-labeled human CRF to its receptor on the cellular membrane of AtT-20 cells, a mouse pituitary cell line, or cells derived from the AtT-20 parental cell line. The assay is a competitive binding radio-receptor assay (RRA) that can discriminate between human CRF and closely related molecules. Whole cells or homogenized cell membrane preparations may be used in the assay. A competitive binding RRA was developed using 100 μl of membrane preparation, 100 μl of radio-labeled human CRF as tracer, and 100 μl of either buffer or competitor. The data obtained is expressed as Percent B/Bo, where B is the corrected CPM for the sample and Bo is the corrected CPM for the total binding tubes (i.e. no competitor).

This bioassay for CRF is based upon the ability of known membrane receptors for CRF to bind ¹²⁵I-Tyr⁰-hCRF and to be displaced by unlabeled competitors. This type of assay is typically called a competitive binding radio-receptor assay (RRA). The unlabeled competitors that are of interest are different batches of hCRF (active drug substance), different lots of formulated drug product that contain hCRF, and CRF-related molecules, such as potential impurities in the active drug substance and known degradation products. Based upon the published literature, various cell lines have been found to express one or more of the CRF receptor subtypes and have been used to measure the effects of CRF, CRF-related peptides, and various agonists and antagonists. For example, AtT-20 cells, a mouse anterior pituitary cell line, has been reported to express only CRF R1, and when CRF binds, an accumulation in intracellular cAMP and an increase in ACTH secretion are observed.

The physiologic effects of CRF are mediated by two G-protein coupled receptors which are the products of two different genes—CRF Receptor Type 1 (CRF R1) and CRF Receptor Type 2 (CRF R2). The two types of receptors share ˜70% sequence homology and both are coupled to adenylate cyclase. However, the two types of receptors have different tissue distributions and bind ligands with different affinities. In addition to CRF, three CRF-related peptides have been discovered in mammals that bind to these receptors: urocortin (Ucn), Ucn II, and Ucn III which is also known as stresscopin. CRF plays a central role in the control of the hypothalamic-pituitary-adrenal axis under stress. Ucn is a 40-amino acid long peptide with 45% sequence homology to CRF that has been cloned from the Edinger-Westphal nucleus, and Ucn II (with 26% sequence homology to CRF) and III have been identified in human and mouse genomic data banks, and all have potent effects on appetite and on the cardiovascular system. All three Ucn's have approximately 10 fold higher affinity for CRF R2 than does CRF, and Ucn II and III are highly selective for CRF R2 since they have little affinity for the CRF R1 subtype. The CRF R2 has at least two and possibly three different splice variants—CRF R2α and CRF R2β and maybe CRF R2γ—which are expressed in different tissues and organs. In rats CRF R2α is predominately found in the brain including the hypothalamus, lateral septum, raphe nuclei of the mid-brain, olfactory bulb, and pituitary. In contrast, CRF R2β is predominately found in the heart, blood vessels, GI tract, and cardiac and skeletal muscle. In addition to the receptors, a CRF binding protein has been described that binds native CRF with a higher affinity than do any of the cellular receptors. The CRF binding protein is expressed in the brain and it might function as a regulator of CRF-mediated neurotransmission.

CRF and CRF-related peptides exert their effect through a cAMP-dependent protein kinase (PKA) pathway in the anterior pituitary and in AtT-20 cells. The connection between the changes in the intracellular cAMP concentration and the stimulation of ACTH secretion results from the interaction between cAMP and the concentration of free calcium ion in the cytosol. In these secretory cells, cAMP plays two major roles (1) to increase the influx of calcium ion into the cell which stimulates secretion and (2) to potentiate the effects of the increased intracellular calcium level on the secretory apparatus. CRF is reported to be specific for activation through its interaction with CRF R1 type receptors: as reported in the literature, it does not activate cells through either the CRF R2α or CRF R2β receptor subtypes.

### 6.2.1. Materials Used and Methods Developed

1. Cells used—AtT-20 and Att-20/D16v-F2 cells were purchased from ATCC (total cost=$493.00). During culture expansion, it became apparent that the AtT-20 cells grew not as single cells in suspension or attached but as “clumps of cells” in suspension. It also became apparent that dispensing these clumps evenly into assay tubes was very difficult. Therefore, AtT-20 cells were cloned by limiting dulition, and selected clones that grew as single cells (not as clumps) either in suspension or lightly-attached. The AtT-20 cells were successfully cloned, and 4 different clones were isolated (clones 1A10, 1G4, 2B8, and 2H1) that grew as single cells either lightly attached or in suspension

2. Culture conditions—Initially all the cell lines and clones were grown in 90% DMEM with high glucose, 10% FCIII (HiClone Labs), containing penicillin and streptomycin, and pH adjusted to pH 7.2 with 4 M NaOH in a humidified atmosphere of 5% CO₂. After the first series of binding experiments were not successful, an alternative culture condition was investigated: 45% DMEM with high glucose, 45% Ham's F-12, 10% FCIII, containing penicillin and streptomycin, and pH adjusted to pH 7.2 with 4 M NaOH in a humidified atmosphere of 10% CO₂. When the binding of ¹²⁵I-Tyr⁰-hCRF was assessed on cells grown under these modified conditions, binding was achieved and displacement with unlabeled competitor was also achieved with both ¹²⁵I-Tyr⁰-human CRF and ¹²⁵I-Tyr⁰-ovine CRF as tracer. All subsequent experiments were performed with cells grown under these conditions.

3. Preparation of ¹²⁵I-Tyr⁰-CRF—New lots of human Tyr⁰-CRF (Tyr⁰-hCRF) and ovine Tyr⁰-CRF (Tyr⁰-oCRF) were purchased from Bachem Bioscience (total cost=$842.82). The lyophilized powder was dissolved in 500 μl acetonitrile:water (1:1/v:v=50% AcCN) and aliquoted in 2 μg, 10 μg, 50 μg, and 100 μg portions into tubes containing 5 μl, 10 μl, 50 μl, and 100 μl of 0.1 M sodium phosphate buffer pH 7.2, respectively. The samples were frozen on dry ice and re-lyophilized. Prior to radio-labeling, polypropylene microfuge tubes are coated with 20 μg of iodogen (Pierce Chem Co.) in 20 μl of dichloromethane and dried under vacuum. The radio-iodination reaction is performed in a chemical fume hood equipped with an activated charcoal filter system. Prior to starting the reaction, a 2 μg sample of Tyr⁰-CRF is dissolved in 40 μl of acetonitrile:water (1:3/v:v=25% AcCN), and 0.2 nmol of Tyr⁰-CRF is transferred into an iodogen tube containing 20 μl of 0.1 M sodium phosphate buffer pH 7.2 and 500 μCi of carrier free Na¹²⁵I. The reaction is incubated at room temperature for 15 minutes with occasional mixing before the reaction mixture is transferred to the top of a 5 ml BioGel P-2 desalting column that has been washed and equilibrated with acetic acid:water (1:1/v:v=50% AcOH). The ¹²⁵I-Tyr⁰-CRF is eluted from the column with 50% AcOH and 0.5 ml fractions are collected. The radio-labeled peptide elutes immediately after the void volume of the column in fractions #4 and 5. The two fractions are pooled and the radio-labeled peptide is used without further purification or is purified by reverse phase HPLC if monoiodo-peptide is desired.

4. RP-HPLC purification of ¹²⁵I-Tyr⁰-hCRF—A C₈ or C₁₈ RP-HPLC column is thoroughly equilibrated with 0.1% TFA in water; a 100 μl aliquot of the pooled radio-labeled peptide obtained from the desalting column is diluted to 1.0 ml with D. H₂0 and immediately transferred to a 2.0 ml injection loop on a manual Rheodyne HPLC injector; and the flow thorough the injector is changed to inject the diluted ¹²⁵I-Tyr⁰-hCRF onto the column. After the column is loaded, a linear gradient program of 0% to 80% acetonitrile in water over 40 minutes is started to elute the bound peptide; the radioactive flow counter is started; and fractions are collected for 0.5 min.

5. Competitive binding radio-receptor assay for CRF using cell membrane preparations—Isolated membrane preparations, since the CRF R1 receptor is associated with the cellularcan, can be used in the assays. Cells are grown and isolated from T-75 flasks as described above, except after collection by centrifugation, they are resuspended in PBS with 1% BSA that contains 20 μg/ml aprotinin (Serologics) as a general protease inhibitor since when the cells are homogenized a number of intracellular proteases will be released.

The cell pellet is resuspended in a small volume (1.5-2.0 ml) of ice-clod PBS with 1% BSA and 20 μg/ml aprotinin, transferred to a 15 ml glass Dounce homogenizer fitted with a tight pestle on ice, and homogenized by 15 strokes with grinding. The lysed cells are transferred to microfuge tubes and centrifuged at 16,000×g for 15 min at 4° C. to collect the particulate membrane fraction. The supernatant is discarded, the particulate fraction is washed by resuspending it in the same ice-cold buffer, and collecting the washed particulate membrane fraction by centrifugation. The membrane fraction is resuspended to a volume equal to 5×10⁶ cells per ml based upon the number of cells originally isolated and homogenized.

The assay reaction is set up as described above (see 5.b) except 100 μl of suspended particulate fraction is used instead of whole cells and the buffer is PBS with 1% BSA and 20 μg/ml aprotinin. The protease inhibitor is included to protect the labeled tracer and competitors from degradation during the overnight incubation. The use of the particulate fraction has improved the reproducibility of the assay in which the displacement of trace by unlabeled CRF is measured in the particulate fraction obtained from the 4 different clones we isolated.

Once the RRA was performing as originally expected, it was tested using the same three competitors used in previous experiments on this project. A cell membrane preparation from clone 1A10 was prepared as described above and tested for its ability to discriminate between different molecules by displacement of the radio-labeled tracer. Concentrations ranging from 10 ng/tube to 3160 ng/tube of human CRF, ovine CRF and the unrelated peptide VIP were assayed for their ability to displace the ¹²⁵I-Tyr⁰-human CRF tracer from its bound membrane association.

In accordance with the invention, the CRF conjugates of the invention have one or more of the biological activities of unmodified CRF, e.g. the ability to competitively bind to the CRF receptor. However, the CRF conjugates may demonstrate differing levels of activity to unmodified CRF.

### 6.3 Determination of the Pharmacokinetic Profile of CRF Conjugates

The CRF conjugates of the invention have an improved pharmacokinetic profile as compared to unmodified CRF. The CRF conjugates of the invention may show an improvement in one or more parameters of the pharmacokinetic profile, including AUC, Cₘₐₓ, clearance (CL), half-life, and bioavailability as compared to unmodified CRF. The following is an example of the determination of the pharmacokinetic profile of unmodified CRF when administered subcutaneously and intravenously.

The objective of this study was to determine the plasma concentration time profile of hCRF following a single intravenous and a single subcutaneous injection in three groups of Sprague-Dawley Crl:CD® :R rats. Concentrations of hCRF in the vehicle (5% mannitol/20 mM pH 4.0 acetate buffer) were 10,100, and 1,000 μg/ml. A dosage volume of 1 ml/kg for all groups resulted in administered doses of 10,100, and 1,000 μg/kg of hCRF for all three dose groups. For the intravenous portion of the study, each of the three dose groups consisted of 72 males. Each of these groups was divided into three sets of replicates. Seven days after the intravenous portion of the study, 61 of the 72 animals from each of the three dose groups were randomly selected for the subcutaneous portion of the study. Each dose group was divided into three sets of replicates. Blood samples were taken at multiple time points via orbital sinus bleeding. Following intravenous dosing, blood samples were collected at time points out to 24 hours post dose. Following subcutaneous dosing, blood samples were collected at time points out to 48 hours post dose. Blood samples were collected from three rats in each dose group for each time point. One animal in the 10 μg/kg group died during the blood collection following the intravenous administration of hCRF. All of the surviving animals were euthanized on the third day following subcutaneous dosing.

Plasma samples were prepared and hCRF concentrations in the plasma samples were determine by an ELISA method. The clearance of intravenously administered hCRF in the rat followed a single exponential pattern and the half-lives were determined to be 9.2, 20.7 and 26.7 minutes for doses of 10,100, and 1,000 μg/kg, respectively. The pharmacokinetics of hCRF administered either intravenously or subcutaneously is dose proportional between 100 and 1,000 μg/kg. At the 10 μg/kg intravenous dose level, the measured hCRF in plasma concentrations approached the detection limits of the ELISA assay. Pharmacokinetic analyses were conducted for this dose group using the limited data obtained. The pharmacokinetic values for the 10 μg/kg intravenous dose group differ from those for the 100 and 1,000 μg/kg groups. This may be a function of the limitations of the ELISA assay at these low levels, and/or may be due to the saturation of potential binding sites for hCRF at the higher doses.

The bioavailability of the subcutaneously administered hCRF at dose level of 100 and 1,000 μg/kg was calculated to be 41% and 37% respectively. In the 10 μg/kg subcutaneous dose group, the measured plasma concentrations were relatively low and approached the detection limit of the assay. A summary of several pharmacokinetic parameters is presented in Table 1 below.

TABLE 1

| Dose | | AUC | Cₘₐₓ | CL | t_{1/2}^{α} | t_{1/2}^{β} | Bioavail. |
|---|---|---|---|---|---|---|---|
| (μg/kg) | | (μg/ml-min) | (ng/ml) | (ml/min/kg) | (min) | (terminal min) | % |
| 10 | (IV) | 0.33 ± 0.03 | 25.1 ± 3.8 | 30.00 ± 3.06 | 9.22 ± 1.05 | NA | NA |
| 100 | (IV) | 30.97 ± 2.50 | 1036.6 ± 105.1 | 3.23 ± 0.26 | 20.71 ± 0.92 | NA | NA |
| 1,000 | (IV) | 292.80 ± 15.58 | 7604.2 ± 488.3 | 3.42 ± 0.18 | 26.69 ± 0.62 | NA | NA |
| 100 | (SC) | 12.86 ± 1.67 | 108.2 ± 16.7 | 7.78 ± 1.01 | 6.96 ± 3.44 | 62.6 ± 5.9 | 41 ± 6 |
| 1000 | (SC) | 107.57 ± 12.18 | 618.1 ± 78.9 | 9.30 ± 1.05 | 21.56 ± 8.15 | 72.1 ± 14.8 | 37 ± 5 |

1. A CRF conjugate comprising CRF, wherein the CRF is chemically modified with polyethylene glycol.

2. The conjugate of claim 1, wherein the CRF has the sequence identified as human CRF.

3. The conjugate of claim 2, wherein the sequence of CRF has been modified to include a cysteine residue.

4. (canceled)

5. The conjugate of claim 3, wherein the cysteine residue has been included at the amino terminus of CRF.

6. The conjugate of claim 3, wherein the cysteine residue has been included at the carboxy terminus of CRF.

7. The conjugate of claim 3, wherein the polyethylene glycol is covalently bound via the cysteine residue.

8. (canceled)

9. The conjugate of claim 5, wherein the sequence of CRF has been modified to include a second cysteine residue, wherein the second cysteine residue has been included at the carboxy terminus of CRF.

10. The conjugate of claim 9, wherein the polyethylene glycol is covalently bound to both cysteine residues.

11. The conjugate of claim 2, wherein the polyethylene glycol is covalently bound via a lysine residue.

12. The conjugate of claim 1, wherein the conjugate has a longer in vivo circulating half-life as compared to unmodified CRF.

13. The conjugate of claim 1, wherein the conjugate has a higher AUC as compared to unmodified CRF.

14. The conjugate of claim 1, wherein the conjugate has a higher bioavailability as compared to unmodified CRF.

15. A pharmaceutical composition comprising CRF chemically modified with polyethylene glycol and a pharmaceutically acceptable diluent, adjuvant or carrier.

16. A method of treating edema in a patient comprising administering to the patient a composition comprising CRF chemically modified with polyethylene glycol and a pharmaceutically acceptable diluent, adjuvant or carrier.

17. The method of claim 16, wherein the composition is administered subcutaneously.

18. The method of claim 16, wherein the composition is administered intravenously.

19. The method of claim 16, wherein the composition is administered once a day.

20. The method of claim 16, wherein the composition is administered at a dose from 0.1 to 5 mg.

21. The method of claim 16, wherein the composition is administered at a dose from 1 to 2 mg.

22. The method of claim 16, wherein the composition is administered at a dose of about 1 mg.

23.-32. (canceled)


SEQUENCE LISTING (3 sequences)

SEQ ID NO: 1 - protein, 41 aa, Homo
  Amino Acid sequence of human CRF peptide
SEEPPISLDLTFHLLREVLEMARAEQLAQQAHSNRKLMEII

SEQ ID NO: 2 - protein, 41 aa, Rattus; identical to SEQ ID NO: 1
  Amino Acid sequence of rat CRF peptide

SEQ ID NO: 3 - protein, 41 aa, Ovis
  Amino Acid sequence of ovis CRF peptide
SQEPPISLDLTFHLLREVLEMTKADQLAQQAHSNRKLLDIA